(19) United States
(12) Patent Application Publication
(10) Pub. No.: US 2010/0179066 A1
(43) Pub. Date: Jul. 15, 2010

(54) Method for Assessing the Risk of a Cardiovascular Disease and for Diagnosing Dyslipidemia

(75) Inventors: Martin John Chapman, Saint Maur (FR); Anatol Kontush, Paris (FR)

Correspondence Address: MCANDREWS HELD & MALLOY, LTD, 500 WEST MADISON STREET, SUITE 3400, CHICAGO, IL, 60661, US

(73) Assignee: INSERM (INSTITUT NATIONAL DE LA SANTÉ ET DE LA REC, Paris (FR)

(21) Appl. No.: 12/602,690

(22) PCT Filed: Jun. 5, 2008
(86) PCT No.: PCT/EP2008/057023

§ 371 Date: Dec. 2, 2009

(30) Foreign Application Priority Data
Jun. 7, 2007 (EP) 07301089.4

(51) Int. Cl. C40B 30/00; G01N 33/92; G01N 33/53

(52) U.S. Cl. 506/7; 436/71; 436/501

(57) ABSTRACT

The invention relates to a method for assessing the risk of a cardiovascular disease and/or for diagnosing dyslipidemia in a patient, said method comprising: a) isolating in a blood sample obtained from said patient the high density lipoprotein (HDL) fraction or a subfraction thereof; and b) measuring in said HDL fraction or subfraction thereof the concentration of one or more biomarkers selected from the group consisting of Sphingosine-1-Phosphate (S1P), sphingomyelin (SM) and Apolipoprotein A-I (apoA-1).

### FIELD OF THE INVENTION

The invention relates to a method for assessing the risk of a cardiovascular disease and/or dyslipidemia.

### BACKGROUND OF THE INVENTION

Cardiovascular diseases are among the leading causes of morbidity and mortality in developed countries, making the prevention of those afflictions a major concern of public health. According to the recent estimates of the World Health Organization, approximately one-third of all deaths (16.7 million people) around the globe resulted from cardiovascular disease in 2002 (World Health Organization, 2004). One of the steps towards the prevention of those disorders is the detection of individuals at risk.

Atherosclerosis represents the major cause for cardiovascular disease. Atherosclerosis can be considered to be a form of chronic inflammation resulting from interaction between modified lipoproteins, monocyte-derived macrophages, T cells and the normal cellular elements of the arterial wall. Atherosclerosis is initiated by accumulation of lipoprotein particles in the extra-cellular matrix of the vessel. The principal lipid components of lipoprotein particles are cholesterol, triglycerides and phospholipids. Cholesterol exits in various forms in the circulation and the major component is low-density lipoprotein cholesterol (LDL-C, approximately 60% of total serum cholesterol), with about 25% in the form of high-density lipoprotein cholesterol (HDL-C) and the remainder circulating in very low-density lipoprotein cholesterol (VLDL-C) and other lipoprotein particles.

Therefore detection of abnormalities of lipid levels in blood or plasma represents a common tool for assessing the risk of atherosclerosis and cardiovascular diseases. Typically, risk factors for atherosclerosis in patients are monitored on blood samples such as measurements of Total Cholesterol, HDL-C, LDL-C, and triglycerides ratios. For example, low circulating levels of HDL-C constitute a major independent and predictive cardiovascular risk factor; in contrast, elevated HDL-C concentrations may be atheroprotective (Rader D J. 2006).

However, from common knowledge, detection of abnormalities of lipid levels does not provide ultimately an efficient tool for predicting cardiovascular risk. Therefore, some biomarkers have been thought to offer a more detailed risk of cardiovascular disease. Currently, biomarkers which may reflect a higher risk of cardiovascular disease include: Higher fibrinogen and PAI-1 blood concentrations, Elevated homocysteine, or even upper half of normal, Elevated blood levels of asymmetric dimethylarginine, High inflammation as measured by C-reactive protein. However, the clinical value of these biomarkers is questionable (Wang T J, et al. 2006). For example numerous cardiovascular disorders can occur in individuals which are not considered at risk relatively to those markers.

Therefore, there is a need for new biochemical markers liable to yield a more accurate prediction of cardiovascular risks.

### SUMMARY OF THE INVENTION

The invention relates to a method for assessing the risk of a cardiovascular disease in a patient, said method comprising:
a) isolating in a blood sample obtained from said patient the high density lipoprotein (HDL) fraction or a subfraction thereof; and
b) measuring in said HDL fraction or subfraction thereof the concentration of one or more biomarkers selected from the group consisting of Sphingosine-1-Phosphate (S1P), sphingomyelin (SM) and Apolipoprotein A-I (apoA-I).
   Another object of the invention relates to a method for diagnosing a dyslipidemia in a patient, said method comprising:
a) isolating in a blood sample obtained from said patient the high density lipoprotein (HDL) fraction or a subfraction thereof; and
b) measuring in said HDL fraction or subtraction thereof the concentration of one or more biomarkers selected from the group consisting of Sphingosine-1-Phosphate (S1P), sphingomyelin (SM) and Apolipoprotein A-I (apoA-I).
   Another object of the invention relates to a method for monitoring the therapeutic outcome of a patient following a therapy for treating a cardiovascular disease and/or dyslipidemia which comprises performing the method as above defined during the therapy.
   Another object of the invention relates to a kit comprising at least 2 antibodies selected from the group consisting of an anti-apoA-I antibody, an anti-S1P antibody and an anti-SM antibody.

### DETAILED DESCRIPTION OF THE INVENTION

### Definitions

The term “S1P” refers to the Sphingosine-1-phosphate. Sphingosine-1-phosphate has been described for example in Zhang et al (1991).

The term “SM” refers to the sphingomyelin. Sphingomyelin has been described for example in (Stoffel W., 1971).

The term “ApoA-I” has its general meaning in the art and refers to the Apolipoprotein A-I, the major component of high density lipoprotein (HDL) in plasma (Rumsey et al., 1994). The term may include any naturally occurring ApoA-I and variants and modified forms thereof. ApoA-I can be from any source, but typically is a mammalian (e.g., human and non-human primate) ApoA-I, particularly a human ApoA-I. Exemplary human native ApoA-I amino acid sequence is provided in GenPept database under accession number NM_{—}000039.

The term “anti-apoA-I antibody” refers to an antibody or a fragment thereof which recognizes apoA-I.

The term “anti-S1P antibody” refers to an antibody or a fragment thereof which recognizes S1P.

The term “anti-SM antibody” refers to an antibody or a fragment thereof which recognizes SM.

The term “cardiovascular disease” has its general meaning in the art and is used to classify numerous conditions that affect the heart, heart valves, blood, and vasculature of the body. Cardiovascular diseases include endothelial dysfunction, coronary artery disease, angina pectoris, myocardial infarction, atherosclerosis, congestive heart failure, hypertension, cerebrovascular disease, stroke, transient ischemic attacks, deep vein thrombosis, peripheral artery disease, cardiomyopathy, arrhythmias, aortic stenosis, and aneurysm. Such diseases frequently involve atherosclerosis. In a preferred embodiment of the invention, the cardiovascular disease is a cardiovascular disease associated with atherosclerosis.

The term “dyslipidemia” refers to a disorder of lipoprotein metabolism, including lipoprotein overproduction or deficiency. Dyslipidemias may be manifested by elevation of the total cholesterol, LDL-C and the triglyceride concentrations, and a decrease in the HDL-C concentration in the blood. In a preferred embodiment of the invention, the dyslipidemia is an atherogenic dyslipidemia.

As used herein, the term “HDL fraction or a subfraction thereof” includes lipoprotein complexes with a density from about 1.06 to about 1.21 g/mL. Lipoproteins may be preparatively fractionated by isopycnic density gradient ultracentrifugation from serum or EDTA plasma as described for example in Chapman et al. 1981 and Guerin et al 2002. Five major subfractions of HDL may be isolated, i.e. large, light HDL2b (d 1.063-1.090 g/mL) and HDL2a (d 1.090-1.120 g/mL), and small, dense HDL3a (d 1.120-1.150 g/mL), HDL3b (d 1.150-1.180 g/mL), and HDL3c (d 1.180-1.210 g/mL). The validity and reproducibility of this density fractionation of HDL particle subspecies has been extensively documented (cf. Chapman et al. 1981 and Guerin et al 2002). The HDL3 subfraction has a density range of 1.120 to 1.210 g/mL. Virtually all HDL particles are known to contain the Apolipoprotein A-I (ApoA-I) as the major component.

As used herein, the term “biomarker” is a biological compound that may be isolated from, or measured in the biological sample and which is differentially present in a sample taken from a subject having established or potentially clinically significant cardiovascular disease and/or dyslipidemia as compared to a comparable sample taken from an apparently normal subject that is not affected with a cardiovascular disease and/or dyslipidemia.

As used herein, the term “predetermined value” refers to the amount of one or more biomarkers in biological samples obtained from the general population or from a select population of subjects. For example, the select population may be comprised of apparently healthy subjects, such as individuals who have not previously had any sign or symptoms indicating the presence of cardiovascular disease and/or dyslipidemia. In another example, the predetermined value may be comprised of subjects having established cardiovascular disease. The predetermined value can be a cut-off value, or a range. The predetermined value can be established based upon comparative measurements between apparently healthy subjects and subjects with established cardiovascular disease and/or dyslipidemia.

As used herein, the term “patient” denotes a mammal, such as a rodent, a feline, a canine, and a primate. Preferably, a patient according to the invention is a human.

The term “blood sample” as used herein refers to a blood sample obtained for the purpose of in vitro evaluation. Examples of blood samples are a whole blood sample, a plasma or a serum sample. As the skilled artisan will appreciate, any such assessment is made in vitro.

### Diagnostic Methods and Kits

The present invention relates to a method for assessing the risk of a cardiovascular disease in a patient, said method comprising:
a) isolating in a blood sample obtained from said patient the high density lipoprotein (HDL) fraction or a subfraction thereof; and
b) measuring in said HDL fraction or subfraction thereof the concentration of one or more biomarkers selected from the group consisting of Sphingosine-1-Phosphate (S1P), sphingomyelin (SM) and Apolipoprotein A-I (apoA-I).
   In a preferred embodiment, the cardiovascular disease is atherosclerosis.
   The invention also relates to a method for diagnosing a dyslipidemia in a patient, said method comprising:
a) isolating in a blood sample obtained from said patient the high density lipoprotein (HDL) fraction or a subfraction thereof; and
b) measuring in said HDL fraction or subfraction thereof the concentration of one or more biomarkers selected from the group consisting of Sphingosine-1-Phosphate (S1P), sphingomyelin (SM) and Apolipoprotein A-I (apoA-I).
   In one embodiment of the invention said biomarker is S1P or said one or more biomarkers are S1P and SM. In a preferred embodiment said one or more biomarkers are S1P, SM and apoA-I.
   In one embodiment of the invention said HDL subfraction is the HDL3 subfraction or a HDL subfraction selected from the group consisting of HDL3a, HDL3b and HDL3c.
   In a preferred embodiment said HDL3 subfraction is the HDL3c subfraction.
   In a further embodiment, the method of the invention further comprises a step consisting in calculating the S1P/SM, S1P/apoA-I or SM/apoA-I ratio.

Isolation of HDL fraction or subfraction thereof may be performed by any known method in the art. For example HDL fraction or subfraction thereof may be prepared by density ultracentrifugation, as described in Mendez, A. J (1991), from plasma or serum. Other methods are also described in Chapman M J. et al. (1981), Guerin M. et al. (2001 and 2002), Rainwater D L. et al. (1998), Cheung M C et al. (1987), Duriez P et al. (1999), Li Z et al. (1994), and Asztalos B F. et al. (1993).

In a particular embodiment, isolation of HDL fraction or subfraction thereof may be performed with a binding partner capable of selectively interacting with apoA-I. HDL is indeed known to contain high amounts of Apolipoprotein A-I (Curtiss L K et al. 2000). For example, the binding partner may be generally an anti-apoA-I antibody that may be polyclonal or monoclonal or a fragment of an antibody. In another example, the binding partner may be an aptamer. The binding partners may be specific for conformation epitopes of apoA-I and thereby allow the isolation of specific HDL subfraction. Examples of binbing partners are described in Curtiss L K et al. (2000), Bustos P. et al. (2000), Marcel Y L. et al. (1989), McVicar J P et al. (1984), Miyazaki O. et al. (2000) and Fielding P E et al. (1994).

Once the HDL fraction or subfraction thereof is isolated, the concentration of one or more of the biomarkers selected from the group consisting of S1P, SM and apoA-I may be then measured by any known method in the art.

For example, the concentration of the biomarker may be measured by using standard immunodiagnostic techniques, including immunoassays such as competition, direct reaction, or sandwich type assays. Such assays include, but are not limited to, Western blots; agglutination tests; enzyme-labeled and mediated immunoassays, such as ELISAs; biotin/avidin type assays; radioimmunoassays; immunoelectrophoresis; immunoprecipitation, gas chromatography, high performance liquid chromatography (HPLC), size exclusion chromatography, solid-phase affinity, etc.

In a particular embodiment, such methods comprise contacting the biological sample with a binding partner capable of selectively interacting with the biomarker present in the HDL fraction or subfraction thereof. The binding partner may be an antibody that may be polyclonal or monoclonal, preferably monoclonal. In another embodiment, the binding partner may be an aptamer.

Polyclonal antibodies of the invention or a fragment thereof can be raised according to known methods by administering the appropriate antigen or epitope to a host animal selected, e.g., from pigs, cows, horses, rabbits, goats, sheep, and mice, among others. Various adjuvants known in the art can be used to enhance antibody production. Although antibodies useful in practicing the invention can be polyclonal, monoclonal antibodies are preferred.

Monoclonal antibodies of the invention can be prepared and isolated using any technique that provides for the production of antibody molecules by continuous cell lines in culture. Techniques for production and isolation include but are not limited to the hybridoma technique originally described by Kohler and Milstein (1975); the human B-cell hybridoma technique (Cote et al., 1983); and the EBV-hybridoma technique (Cole et al. 1985). Alternatively, techniques described for the production of single chain antibodies (see e.g. U.S. Pat. No. 4,946,778) can be adapted to produce single chain antibodies. Antibodies useful in practicing the present invention also include fragments including but not limited to F(ab′)₂ fragments, which can be generated by pepsin digestion of an intact antibody molecule, and Fab fragments, which can be generated by reducing the disulfide bridges of the F(ab′)₂ fragments. Alternatively, Fab and/or scFv expression libraries can be constructed to allow rapid identification of fragments having the desired specificity. For example, phage display of antibodies may be used. In such a method, single-chain Fv (scFv) or Fab fragments are expressed on the surface of a suitable bacteriophage, e.g., M13. Briefly, spleen cells of a suitable host, e.g., mouse, that has been immunized with a protein are removed. The coding regions of the VL and VH chains are obtained from those cells that are producing the desired antibody against the protein. These coding regions are then fused to a terminus of a phage sequence. Once the phage is inserted into a suitable carrier, e. g., bacteria, the phage displays the antibody fragment. Phage display of antibodies may also be provided by combinatorial phage may then be used as part of an immunoassay.

Examples of antibodies against apoA-I are described in Curtiss L K et al. (2000), Bustos P. et al. (2000), Marcel Y L. et al. (1989), McVicar J P et al. (1984), Miyazaki O. et al. (2000) and Fielding P E et al. (1994).

Examples of antibodies against S1P and SM are described in Visentin B et al. (2006) and Strejan G H. et al. (1979) respectively.

Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L., 1990. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S. D., 1999. Peptide aptamers consist of conformationally constrained antibody variable regions displayed by a platform protein, such as E. coli Thioredoxin A, that are selected from combinatorial libraries by two hybrid methods (Colas et al., 1996).

The aforementioned assays may involve the binding of the binding partner (ie. Antibody or aptamer) to a solid support. Solid supports which can be used in the practice of the invention include substrates such as nitrocellulose (e.g., in membrane or microtiter well form); polyvinylchloride (e.g., sheets or microtiter wells); polystyrene latex (e.g., beads or microtiter plates); polyvinylidine fluoride; diazotized paper; nylon membranes; activated beads, magnetically responsive beads, and the like.

The binding partners of the invention such as antibodies or aptamers may be labelled with a detectable molecule or substance, such as a fluorescent molecule, a radioactive molecule or any others labels known in the art. Labels are known in the art that generally provide (either directly or indirectly) a signal.

As used herein, the term “labeled”, with regard to the antibody or aptamer, is intended to encompass direct labeling of the antibody or aptamer by coupling (i.e., physically linking) a detectable substance, such as a radioactive agent or a fluorophore (e.g. fluorescein isothiocyanate (FITC) or phycoerythrin (PE) or Indocyanine (Cy5)) to the antibody or aptamer, as well as indirect labeling of the probe or antibody by reactivity with a detectable substance. An antibody or aptamer of the invention may be labeled with a radioactive molecule by any method known in the art. For example radioactive molecules include but are not limited radioactive atom for scintigraphic studies such as I123, I124, In111, Re186, Re188.

In a particular embodiment, an ELISA method may be suitable for measuring concentration of the biomarker, wherein the wells of a microtiter plate are coated with a set of antibodies against the biomarker. The HDL fraction or subfraction thereof containing or suspected of containing the biomarker is then added to the coated wells. After a period of incubation sufficient to allow the formation of antibody-antigen complexes, the plate(s) can be washed to remove unbound moieties and a detectably labeled secondary binding molecule added. The secondary binding molecule is allowed to react with any captured sample marker protein, the plate washed and the presence of the secondary binding molecule detected using methods well known in the art.

In another particular embodiment, the isolation of HDL fraction or subfraction thereof and measuring concentration of the biomarker may be performed with an array chip. Such an array technology allows a large number of experiments to be performed simultaneously on a single substrate, commonly known as a biochip when used for biological analytes. Example of array chips are described in the international patent document WO2007012885 and Dupuy A M. et al. (2005), Weinberger S R et al. (2000) and Jain K K et al. (2000).

For example the binding partner for apoA-I may be immobilized at the surface of said array chip. The biological sample obtained from said patient is then deposited in the array chip. After a period of incubation sufficient to allow the formation of antibody-apoA-I complexes, the array chip is then washed to remove unbound moieties, and thus allowing the isolation of HDL fraction or subfraction thereof. In a second step, the measurement of the biomarker (ie. S1P, SM or apoA-I) concentration may be performed with a second binding partner specific for said biomarker. In a preferred embodiment, said binding partner is labelled thus allowing the formation of a set of “spots” (coloured deposit) specific for the biomarker. For example, detection and quantification may be performed by analysing the spots in said array chip with a specific detector.

Alternatively, the method of the invention further may comprise a step of comparing the concentration of the biomarker with a predetermined threshold value. Said comparison is indicative of the risk of cardiovascular disease in said patient or that the patient is affected with dyslipidemia.

The method of the invention may be thus useful for classifying patients affected by cardiovascular disease and then may be used to choose the accurate treatment for said patient. For example, patients with a high risk score may receive a more intensive treatment and attention compared to patient with a weak risk score. Such a method may thus help the physician to make a choice on a therapeutic treatment which can accordingly consist in administering accurate drugs to the patients. Costs of the treatments may therefore be adapted to risk of the patients.

The method of the invention may be used in combination with traditional methods used to assess the risk of cardiovascular disease and/or to diagnose dyslipidemia in a patient.

In a further aspect, the method of the invention may be applied for monitoring the therapeutic outcome of a patient following a therapy for treating a cardiovascular disease and/or dyslipidemia. Actually, innovative pharmacological approaches to raise anti-atherogenic high-density lipoprotein-cholesterol (HDL-C) are currently of considerable interest, particularly in atherogenic dyslipidemias characterized by low levels of HDL-C, such as type 2 diabetes, the metabolic syndrome, and mixed dyslipidemia, but equally among individuals with or at elevated risk for premature cardiovascular disease. The available options for elevating low HDL-C levels have been recently reviewed by Ashen and Blumenthal (2005) and include but are not limited to Niacin, Cholesteryl Ester Transfert Protein (CETP) inhibitors (such as JTT-705 and Torcetrapib), agonists of peroxisome proliferator-activated receptors (PPAR)-alpha (such as fibrates), agonists of PPAR-gamma, statins, apoA-I mimetic peptides, reconstituted HDL, CB1 agonists and MK-0859. (Chapman M J. et al. 2006, and Kontush A, et al. 2006). Therefore methods of the invention may be suitable to investigate whether the treatment administered to the patient is effective.

Methods of the invention are particularly suitable for assessing the residual cardiovascular risk after a lipid lowering treatment to reduce plasma LDL-C levels, in particular after treatment with a statin.

A further object of the invention relates to the use of S1P, SM or apoA-I thereof as a marker of a dyslipidemia or a cardiovascular disease, in particular atherosclerosis.

Yet another object of the invention relates to a kit comprising at least 2 binding partners selected from the group consisting of a binding partner which selectively interacts with apoA-I, a binding partner which selectively interacts with S1P and a binding partner which selectively interacts with SM.

For example the kit may include a binding partner which selectively interacts with S1P and a binding partner which selectively interacts with SM. The kit may also include a binding partner which selectively interacts with apoA-I, a binding partner which selectively interacts with S1P and a binding partner which selectively interacts with SM.

Yet another object of the invention relates to a kit for performing methods of the invention, comprising at least 2 antibodies selected from the group consisting of an anti-apoA-I antibody, an anti-S1P antibody and an anti-SM antibody.

For example the kit may include an anti-S1P antibody and an anti-SM antibody. The kit may also include an anti-apoA-I antibody, an anti-S1P antibody and an anti-SM antibody.

In a particular embodiment, the antibodies are labelled as above described. The kit may also contain other suitably packaged reagents and materials needed for the particular detection protocol, including solid-phase matrices, if applicable, and standards.

The invention will further be illustrated in view of the following FIGURE and example.

### FIGURE LEGEND

FIG. 1. Weight % content of SM (open bars) and molar content of S1P (filled bars) in HDL subfractions from normolipidemic subjects (n=6). SM is expressed as weight % of the sum of all molecular species of lipids measured by HPLC (PL, CE, FC, lysophosphatidylcholine and S1P). For superscripts, see Table 1

### EXAMPLE

### Material & Methods

Blood samples: Serum and EDTA plasma (final EDTA concentration 1 mg/ml) were prepared from venous blood collected into sterile evacuated tubes (Vacutainer) from nine healthy male volunteers after an overnight fast. Donors were normolipidemic, non-obese, normotensive, normoglycemic and displayed normal levels of hsCRP and 8-isoprostanes (Nobecourt E, et al. 2004, Hansel B. et al. 2004).

TABLE 1
Biological characteristics of normolipidemic subjects
(n = 9)

| | |
|---|---|
| Age (y) | 48 ± 16 |
| Sex (M/F) | 9/0 |
| Waist circumference (cm) | 81 ± 6 |
| BMI (kg/m²) | 22.8 ± 1.5 |
| SBP (mmHg) | 130 ± 12 |
| DBP (mmHg) | 86 ± 8 |
| TC (mg/dl) | 170 ± 25 |
| TG (mg/dl) | 83 ± 14 |
| LDL-C (mg/dl) | 100 ± 24 |
| ApoB-100 (mg/dl) | 86 ± 19 |
| VLDL-C (mg/dl) | 17 ± 4 |
| HDL-C (mg/dl) | 53 ± 9 |
| ApoA-I (mg/dl) | 155 ± 14 |
| Glucose (mg/dl) | 95 ± 14 |
| CRP (mg/l) | 0.8 (0.2; 1.5) |
| 8-Isoprostanes (ng/l) | 39 ± 22 |

None of our blood donors was receiving antioxidant vitamin supplementation or drugs known to affect lipoprotein metabolism; all subjects were non-smokers and either abstainers or only moderate alcohol consumers. After blood collection, serum and EDTA plasma were immediately separated by centrifugation at 4° C.; plasma and serum were each mixed with sucrose (final concentration 0.6%) as a cryoprotectant for lipoproteins (Rumsey S.C., et al. 1994) and frozen at −80° C. under nitrogen for less than 3 months.

Fractionation of lipoproteins: Lipoproteins were preparatively fractionated by isopycnic density gradient ultracentrifugation as previously described (Chapman M J. et al. 1981, Guerin M. et al. 2001 and 2002). Five major subfractions of HDL were isolated, i.e. large, light HDL2b (d 1.063-1.090 g/ml) and HDL2a (d 1.090-1.120 g/ml), and small, dense HDL3a (d 1.120-1.150 g/ml), HDL3b (d 1.150-1.180 g/ml), and HDL3c (d 1.180-1.210 g/ml). Lipoproteins were stored at 4° C. and analysed within 10 days. As LCAT activity rapidly decreases in HDL subfractions upon storage at 4° C., LCAT was assayed not later than 48 h after HDL isolation. Before use, KBr (and, in the case of plasma, EDTA) was removed from LDL and HDL solutions by exhaustive dialysis for 24 h at 4° C.

Physicochemical characterisation of lipoproteins: Total protein, lipids and apolipoproteins. Total protein, total cholesterol (TC), FC, PL and TG contents of isolated lipoprotein subfractions were determined using commercially available enzymatic assays (coefficients of variation <7%) (Kontush A, et al. 2005). Cholesteryl esters (CE) were calculated by multiplying the difference between total and free cholesterol by 1.67 (Chapman et al. 1981). Total HDL mass was calculated as a sum of total protein, FC, PL, TG and CE. ApoA-I, apoA-II, apoC-II, apoC-III and apoE were measured using immunonephelometry (Pastier D. et al. 2001). Plasma levels of LpA-I and LpA-I:A-II were measured by immunoelectrophoresis (Sebia, Issy-les-Moulineaux, France; coefficient of variation, 3%).

Molecular weights of HDL subfractions were calculated by transforming concentration data (mg/dl) into absolute molar units using molecular weights of CE, FC, PL and TG of 650, 387, 750 and 850 Da, respectively (Chancharme L et al. 1999); the HDL protein moiety was considered to consist of two apolipoproteins, apoA-I and apoA-II, and the molecular weight of the protein moiety in each HDL subfraction was calculated using the total protein content (mg/dl) converted to molarity on the basis of relative mass content of apoA-I and apoA-II.

Enzymatic activities: PON1 activity of HDL subfractions (100 μg protein/ml) isolated from serum was determined photometrically in the presence of CaCl2 (1 mM) using phenyacetate or paraoxon as a substrate (Tsimihodimos V et al. 2002; Kontush A et al. 2003). Activity of PAF-AH was assessed using C6NBD phosphatidylcholine as a fluorescent substrate (Kontush A et al. 2003, Gowri M S et al. 1999). LCAT activity was measured using a fluorescent LCAT activity kit (Roar Biomedical, New York, N.Y., USA) (Kontush A et al. 2003). Sphingomyelinase activity was assayed using the Amplex Red fluorescence kit (Invitrogen, Carlsbad, Calif., USA).

Molecular species of cholesteryl esters and phosphatidylcholine: Lipids were extracted with methanol/hexane (4/10 v/v) from aliquots of HDL subfractions as previously described (Chancharme L et al. 2002). Briefly, the hexane layer (upper phase containing CE) and the methanol/water layer (lower phase containing phosphatidylcholine) were separated by centrifugation at 1500 g for 5 min and evaporated to dryness under nitrogen. The dried residue corresponding to phosphatidylcholine was dissolved in methanol and, after loading onto the HPLC system, separation of molecular species of phosphatidylcholine was performed as previously described (Therond P. et al. 2000) with a 250×4.6 mm C18 Kromasil column with 6% ammonium acetate (10 mM, pH5.0)/94% methanol as mobile phase. The dried residue corresponding to CE was dissolved in methanol containing 1% hexane and separation of CE was performed with a 150×4.6 mm C18 Spherisorb column and methanol as mobile phase as previously described (Therond P et al. 2000).

Major classes of C E, P L and lysophosphatidylcholine: Lipids were extracted from HDL subspecies using the method of Folch et al. (1957). Organic extracts were evaporated to dryness under nitrogen and the dried lipids were dissolved in isopropanol:hexane (60:40, v/v). Normal phase HPLC separation was performed on a Kromasil silica 5 μm (2.1 mm i.d.×250 mm) column with elution using a mobile phase of isopropanol: hexane: 25 mM potassium acetate (pH 7.0) (57.5:36:6.5, v/v/v) at 50° C. with a gradient from 36 to 50% of hexane in 30 minutes and a flow rate of 0.4 ml/minute. Chromatographic peaks were identified using UV absorbance at 205 nm. All PL were clearly separated into phosphatidylcholine, phosphatidylethanolamine, sphingomyelin (two peaks corresponding to two molecular species, 16:0 sphingomyelin and 18:0 sphingomyelin), phosphatidylinositol and lysophosphatidylcholine. Individual PL and lysophosphatidylcholine peaks were identified by comparison of retention time to known standards (16:0/18:2 phosphatidylethanolamine, 16:0 sphingomyelin, 16:0/18:2 phosphatidylinositol, 18:0 lysophosphatidylcholine). Calibration curves for each lipid standard were established for quantification in HDL subspecies (coefficients of variation, <5%). The extraction efficacy was similar for all lipids between large and small HDL particles. Indeed, recoveries of minor lipids (phosphatidylinositol, sphingomyelin and lysophosphatidylcholine) from large HDL2 vs. small HDL3 were as follows (n=4): phosphatidylinositol, 90.1±2.8 vs. 89.5±1.9%; sphingomyelin, 91.1±5.5 vs. 91.6±3.5%; lysophosphatidylcholine, 95.3±3.0 vs. 90.3±7.6%. Recoveries of major lipids (phosphatidylcholine and phosphatidylethanolamine) ranged from 91 to 95% and did not differ between large and small HDL either.

Sphingosine-1-phosphate (S1P): S1P was determined in HDL subspecies as described by Nofer et al (2004). Methanol (1 ml) containing 2.5 μl concentrated HCl was added to 100 μL of HDL solution (0.5 to 2.0 mg HDL per ml buffer as a function of HDL subspecies). C17-S1P (15 pmoL) was added as internal standard and lipids were extracted by addition of 1 ml chloroform, 200 μl NaCl (4 M) and 100 μl NaOH (3 M). The alkaline aqueous S1P-containing phase devoid of other sphingoid bases and of the majority of hydrophobic PL was transferred to a clean tube and the organic phase re-extracted with 0.5 ml methanol, 0.5 ml of 1M NaCl and 3N NaOH (50 μl). The alkaline aqueous phases were combined, acidified with 100 μl concentrated HCl and extracted twice with 1.5 ml chloroform. The organic phases were evaporated and the dried lipids were dissolved in a mixture (50 μl) of methanol and 0.07 M K₂HPO₄ (9:1 v/v). A derivatization mixture of 10 mg o-phthaldialdehyde, 200 μl ethanol, 10 μl 2-mercaptoethanol and 10 ml boric acid (3% v/w) was prepared and adjusted to pH 10.5 with KOH. Five μl of the derivatization mixture was added to the lipids and the solution incubated for 15 minutes at room temperature. The derivatives were analyzed with a Hewlett Packard HPLC system using an RP 18 Kromasil column (2.1 mm i.d.×150 mm) maintained at 45° C. Separation was performed with the isocratic eluent containing methanol: K₂HPO₄ (0.07 M) (78:22 v/v) at a flow rate of 0.25 mL/min. The derivatives were detected selectively using a Hewlett Packard spectrofluorometer with an excitation wavelength of 340 nm and an emission wavelength of 456 nm. S1P was quantified by comparison of its fluorescent signal with that of the derivative of the internal standard (coefficients of variation, <5%).

Fatty acids: Fatty acids were analysed by gas-liquid chromatography (GLC) using a Shimadzu instrument (Kyoto, Japan) equipped with a 30-m Supelcowax 10 glass capillary column (Supelco, Bellefonte, Pa., USA) and connected to a Delsi Nermag integrator (Argenteuil, France). Total lipids were extracted according to the method of Bligh and Dyer (Bligh E G. Et al. 1959) from 0.1 ml of each HDL subspecies. The chloroform phase was evaporated to dryness under nitrogen and total lipids were transesterified with 1 ml of methanolic KOH-BF3 at 70° C. for 5 minutes. After extraction by 1 ml of hexane and evaporation under nitrogen, the residue was reconstituted in isooctane (25 μl). Two to four μl of this organic solution of fatty acid methyl esters were separated and measured by GLC. Peaks were identified by comparing the retention time with those of the corresponding standards and quantified by using an internal standard (heptadecanoic acid; coefficient of variation, <5%).

Antioxidative activity of HDL subfractions: Antioxidative activity of HDL subfractions was assessed towards normolipidemic reference LDL.1, 2 LDL (10 mg TC/dl) was oxidised in the absence or presence of HDL particles at 37° C. in Dulbecco's PBS (pH 7.4) by 1 mM 2,2″-azobis-(2-amidinopropane) hydrochloride (AAPH); HDL subfractions were added to LDL immediately before oxidation at a final concentration of 10 mg total mass/dl (Nobecourt E. et al. 2005, Hansel B. et al. 2004). Accumulation of conjugated dienes was measured as the increment in absorbance at 234 nm (Nobecourt E. et al. 2005, Hansel B. et al. 2004). Absorbance kinetics were corrected for the absorbance of AAPH itself run in parallel as a blank. The kinetics of diene accumulation revealed two characteristic phases, the lag and propagation phases. For each curve, the duration of each phase, average oxidation rates within the propagation phase and amount of dienes formed at the end of the propagation phase (maximal amount of dienes) were calculated.

Antiapoptotic activity of HDL subfractions: Human microvascular endothelial cells-1 (HMEC-1) were grown in MCDB 131 medium supplemented with 10% fetal calf serum, glutamine (2 mM), 100 U/ml penicillin and 100 μg/ml streptomycin. At 100% confluence, cells were plated at a concentration of 10,000 to 40,000 cells/ml and subsequently (24 or 48 h later) preincubated in the absence (control) or in the presence of each HDL subfraction (25 μg protein/ml) in a serum-free RPMI medium for the next 24 h. The medium containing HDL was removed, cells washed and incubated with oxidised LDL (oxLDL) for up to 24 hours. LDL (d 1.018-1.065 g/ml; 200 mg apoB-100/dl corresponding to 4 μM LDL) was oxidised by UV irradiation in the presence of Cu²⁺ (5 μM). The LDL preparation was irradiated for 2 h as a thin film (5 mm) in an open beaker placed 10 cm under the UV-C source (HNS 30W OFR Osram UV-C tube, λmax 254 nm, 0.5 mW/cm2). This approach produces mildly oxLDL that selectively induces apoptosis rather than necrosis in endothelial cells (Escargueil-Blanc I. et al. 1997). The level of LDL oxidation was evaluated by the absorbance increment at 234 nm (Δ234 nm), which reflects the accumulation of lipid hydroperoxides possessing a conjugated diene structure; diene concentration in the samples was calculated using ε=29,500 M⁻¹ cm⁻¹(Esterbauer H. et al. 1989). The absorbance increment at 234 nm of 0.050-0.150 was used, corresponding to 4-13 mol of conjugated dienes/mol LDL. After irradiation, oxLDL preparations were sterilised using 0.2 μm Millipore filters.

Apoptosis and necrosis in HMEC-1 were quantified using the Annexin V-FITC/propidium iodide (PI) kit (Beckman Coulter, Roissy, France). Cell medium was collected and cells were washed twice with PBS at 4° C., trypsinised for 15 min at 37° C., centrifuged, washed and stained for 15 min at 4° C. in the dark. The level of primary apoptosis was specifically determined by flow cytometry analysis at excitation and emission wavelengths of 492 and 520 nm respectively for annexin V-FITC and 550 and 680 nm respectively for PI; cells displaying primary apoptosis were identified by a combination of high FITC and low PI signals. Non-stained controls were used to correct for cellular auto-fluorescence in the presence of oxLDL.

To prepare S1P-enriched HDL subfractions, EDTA plasma from a normolipidemic donor was incubated overnight with S1P under constant stirring at 4° C. S1P was added as a methanolic solution to an empty tube (final concentration 5 μM), methanol evaporated and plasma added. Plasma from the same subject incubated in parallel was used as a control. Subsequently, S1P-enriched and control HDL subfractions were isolated and their S1P content measured by HPLC as described above. HMEC-1 (20,000-30,000 cells/well) were plated in 12-well plates and incubated in the absence or presence of each HDL subfraction (25 μg of total protein/ml) in a serum-free RPMI 1634 medium for 24 h; oxLDL (Δ234 nm, 0.050-0.150 corresponding to 4-13 mol of conjugated dienes/mol LDL) was added for 16 to 24 h and flow cytometry performed as described above.

Statistical analysis: Significance of differences in chemical composition between HDL subfractions was analysed with a one-way analysis of variance (ANOVA) followed by a post-hoc Bonferroni correction for multiple group comparisons or with Student's t-test for dependent samples when applicable. Using the correction for multiple comparisons, the p-level for a given comparison was adjusted down in such a way that the alpha level of 0.05 can be still considered significant.

Pearson's moment-product correlation coefficients were calculated to evaluate relationships between variables. All results are expressed as means±SD unless otherwise indicated. Statistical analysis of differences between HDL subfractions was carried out by Wilcoxon's test. Values of P<0.05 were considered significant. Calculations were performed using STATISTICA 6.1 (StatSoft Inc., www.statsoft.com). or StatView (SAS Institute Inc., www.statview.com/) software packages.

Results:

Plasma concentrations and physicochemical characteristics of HDL particle species: On a molar basis, large, light HDL2a and small, dense HDL3a subclasses predominated in normolipidemic subjects; HDL subclass concentrations decreased in the order HDL2a≧HDL3a>HDL2b>HDL3b>HDL3c. Molecular weights and particle diameters diminished in parallel with increment in HDL density from HDL2b to 3c. Progressive reduction in HDL particle size with increase in hydrated density was associated with progressive elevation in protein content and in surface/core ratio; reduction in size was equally accompanied by diminution in core neutral lipid content (<50 mol CE and TG per HDL particle in HDL3b and 3c), consistent with the marked predominance of surface components in small, dense HDL.

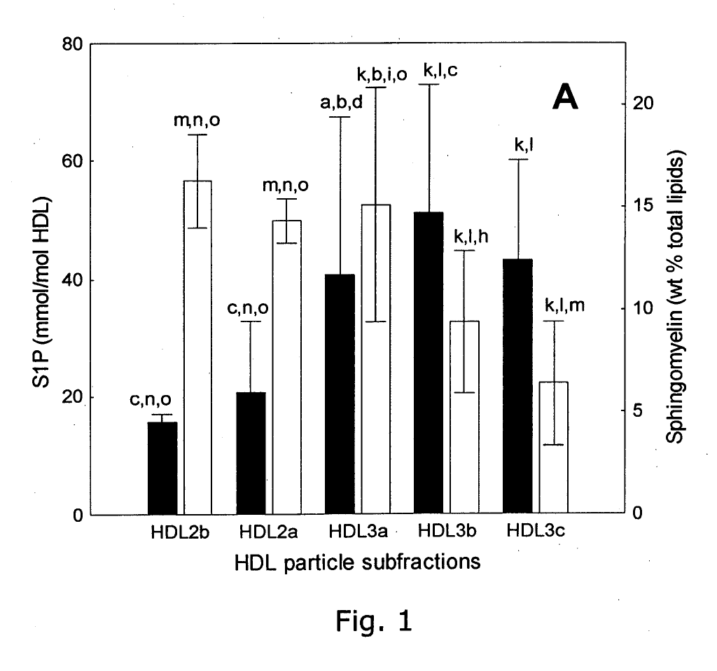

Molecular lipid species in HDL particles: Molar particle content of CE, FC and of PL subclasses including phosphatidylcholine, phosphatidylethanolamine, phosphatidylinositol, SM and lysophosphatidylcholine showed a marked tendency to decrease progressively in parallel with increase in hydrated density from HDL2b to HDL3c. Indeed, particle content of CE, FC and all PL subclasses on a molar basis was significantly lower (p<0.05) in small HDL3b and 3c vs. large HDL2b and 2a particles (Table 2, see below). Furthermore, small HDL3c contained significantly less phosphatidylcholine, phosphatidylethanolamine, phosphatidylinositol, SM, lysophosphatidylcholine and FC relative to HDL3b on a molar basis. Interestingly, no such differences were evident between HDL subspecies when data for CE, phosphatidylcholine, phosphatidylethanolamine, phosphatidylinositol and lysophosphatidylcholine were expressed as a percentage of total lipids (Table 2). Indeed, CE content was maintained in the range 37.9 to 50.5% across HDL subspecies; with respect to PL subclasses, phosphatidylcholine varied from 33.8 to 40.8%, phosphatidylethanolamine from 0.7 to 1.0%, phosphatidylinositol from 2.8 to 5.1% and lysophosphatidylcholine from 0.5 to 0.6%. SM constituted an exception however, as the proportion of this lipid decreased progressively in parallel with HDL density from 12.8% in HDL2b to 6.2% in HDL3c (FIG. 1). Consequently, the SM/phosphatidylcholine ratio decreased from 0.62 in HDL2b to 0.14 in HDL3c, consistent with earlier data (Bagdade J D et al. 1995) in total HDL2 and HDL3. The depletion of SM in small HDL was not associated with elevated sphingomyelinase activity, as no such activity was detected in HDL subfractions, consistent with earlier data (Holopainen J M et al. 2000).

As for SM, FC content decreased twofold from 8.2% in HDL2b to 4.2% in HDL3c (Table 2). Interestingly, the CE/FC ratio significantly increased with HDL density from 4.3±0.6 in HDL2b to 10.2±2.1 in HDL3c (p>0.001).

When HDL particle contents of molecular species of CE and phosphatidylcholine were evaluated, cholesteryl linoleate predominated among CE, with a tendency to enrichment in HDL3c. Similarly, cholesteryl arachidonate, the minor ester, tended to be more abundant in HDL3b and 3c. As in the case of PL subclasses, the absolute molar content of each CE species diminished in parallel with diminution in the molecular weight and particle size from HDL2 to HDL3 subfractions.

The 18:2/16:0, 18:2/18:0 and 20:4/16:0 species of phosphatidylcholine predominated in all HDL particle subclasses, representing 17.3-20.3, 6.7-8.3 and 4.3-6.0% respectively of total lipids. Similarly, percentage content of minor PL species containing arachidonic (20:4/16:0 and 20:4/18:0) and docosahexaenoic (22:6/16:0 22:6/18:0) acids were relatively constant across the HDL particle spectrum (6.8-9.3% and 2.6-3.1% respectively).

When lipid moieties of HDL particle subspecies were analysed on the basis of their total fatty acid content, thereby including all PL, CE and TG fatty acid residues, the % distribution of saturated (SFA), monounsaturated (MUFA) and polyunsaturated (PUFA) n-6 and n-3 fatty acids was indistinguishable between HDL particle subspecies. n-6 PUFAs preponderated in all subfractions (45-50% of total), with lesser abundance of SFA (30-35%), MUFA (12-15%) and n-3 PUFA (<5%). Nothwithstanding such marked similarities in overall fatty acid profile among HDL subfractions, absolute molar contents of fatty acids were significantly lower in small HDL3b and 3c (p<0.05) as compared to the larger HDL2b, 2a and 3a subfractions.

Sphingosine-1-phosphate (S1P): Among the minor bioactive lipid components, the abundance of S1P per HDL particle was asymmetric across the HDL spectrum, with preferential enrichment (40-50 mmol/mol HDL) in HDL3 as compared to HDL2 subfractions (15-20 mmol/mol; FIG. 1). For example, S1P was 3-fold enriched in HDL3c (approximately 1 mole per 17 HDL particles) as compared to HDL2b (approximately 1 mole per 50 HDL particles). By contrast, no considerable difference was detected in the content of S1P between HDL3a, 3b and 3c subfractions. HDL molar content of S1P negatively correlated with weight % content of SM (r=−0.73, p=0.002) and positively correlated with that of apoA-I (r=0.63, p<0.001) and total protein (r=0.70, p<0.001) as well as with the HDL surface-to-core ratio (r=0.70, p<0.001). As a result, the S1P/SM molar ratio increased (p<0.001 for trend) from 0.28±0.96 mmol/mol in HDL2b to 11.0±0.86 mmol/mol in HDL3c.

Protein moieties of HDL particle species: The particle content of apoA-I fell progressively from a maximal level of approximately 4 mol/mol in HDL2 to a particle average of 2.5 mol/mol in HDL3c; HDL subfraction content of LpA-I decreased in a similar manner. By contrast, maximal particle contents of apoA-II occurred in HDL2a and 3a (1.1-1.4 mol/mol), a finding consistent with the predominance of LpA-I:A-II particles in the HDL2a and 3a subfractions, in which the abundance of LpA-1:A-II was two- to threefold greater than that of LpA-I. Interestingly, and consistent with earlier published data (Cheung M C. et al. 1979). the molar ratio of apoA-I to apoA-II was highest in the largest and smallest HDL particles respectively (HDL2b, 5.59±2.09; HDL3c, 6.89±4.23), thereby attesting their preferential apoA-I enrichment. Consistent with these data, the highest ratio of LpA-I/LpA-I:LpA-II was seen in the HDL2b and HDL3c particles, confirming an earlier report (Cheung M C. et al. 1982). LpA-I:LpA-II particles were more abundant than LpA-I in all HDL subfractions (LpA-I/LpA-I:LpA-I ratio <0.67).

Quantitatively minor HDL apolipoproteins including apoC-II, apo C-III and apoE are key determinants of HDL metabolism. While apoC-II was detected in all HDL subclasses, it was preferentially enriched (up to threefold) in large, light HDL2b (0.85 mol/mol vs. 0.24-0.37 mol/mol in other subclasses. ApoC-III was equally enriched in HDL2b (1.5 mol/mol); in contrast to apoC-II however, apoC-III content fell markedly with increase in density, resulting in an elevated apoC-II/apoC-III ratio in small, dense HDL3b and 3c subfractions (1.23 and 3.68 mol/mol respectively). By contrast, no significant difference in the content of apoE was found between HDL subfractions.

Enzymatic activities: PON1 activity of HDL subfractions with phenyl acetate as substrate increased in the order HDL2b<HDL2a<HDL3a<HDL3b<HDL3c. PON1 activity to paraoxon was similarly distributed among HDL subfractions, consistent with earlier data (Valabhji J, et al. 2001). PAF-AH activity was significantly elevated in the large HDL2b and small HDL3c subtractions; LCAT activity tended to be elevated in the HDL3c subfraction consistent with our recent data (Kontush A. et al. 2003).

Antioxidative activity of HDL subfractions: The capacity of isolated HDL subtractions to inhibit LDL oxidation by AAPH increased in the order HDL2b<HDL2a<HDL3a<HDL3b<HDL3c on a particle mass basis, consistent with earlier data (Kontush A. et al. 2003). Small, dense HDL3b and 3c subtractions (but not HDL2) decreased the oxidation rate of LDL in the propagation phase (−27 and −25% respectively) and equally prolonged this phase (+37 and +39% respectively).

The capacity of HDL subfractions to inhibit LDL oxidation was significantly correlated with the S1P/SM molar ratio (r=−0.43, p<0.05, vs. LDL oxidation rate and r=0.58, p<0.01, vs. duration of the propagation phase).

Antiapoptotic activity of HDL subfractions: Death of HMEC-1 endothelial cells treated with mildly oxLDL (200 μg apoB-100/ml; 4-13 mol of conjugated dienes/mol LDL) occurred mainly through an apoptotic process, as suggested by the number of cells exhibiting characteristic morphological nuclear changes, such as chromatin condensation and nuclear fragmentation. By contrast, the level of primary necrosis was very low (<2%) as shown by flow cytometry using the annexin V-FITC/PI test.

Apoptosis and necrosis were quantified as cellular binding of annexin V and staining by PI; indeed, annexin V specifically interacts with phosphatidylserine in the extracellular membrane leaflet of apoptotic cells, whereas PI stains necrotic nuclei (Hanshaw R G. et al. 2005). Preincubation with small, dense HDL3b and 3c (25 μg protein/ml) significantly diminished (−54%, p<0.01, and −148%, p<0.001, respectively) annexin V binding to HMEC-1 induced by oxLDL, whereas large HDL2a and 3a particles were not effective. By contrast, large HDL2b was able to significantly inhibit apoptosis in this assay (−47%, p<0.001), an observation potentially related to their elevated content of neutral phospholipids (PL); such PL might replace phosphatidylserine in the outer plasma membrane, thereby attenuating binding of annexin V.

The capacity of HDL subfractions to inhibit HMEC-1 apoptosis was strongly and positively correlated with the S1P/SM molar ratio (r=0.73, p<0.001). By contrast, no significant correlation between the antiapoptotic and antioxidative activities of HDL subfractions measured as above was found (data not shown).

In a separate experiment, normolipidemic human plasma was preincubated without or with S1P (5 μM). HDL isolated from S1P-supplemented plasma were enriched in S1P (from 1.8-fold in HDL3c to 2.9-fold in HDL2b, n=3). Such S1P enrichment of HDL subfractions consistently enhanced their antiapoptotic activity. Indeed, S1P-enriched HDL2b, 2a and 3a decreased annexin V binding to HMEC-1 by 30, 35 and 24% respectively as compared to their non-enriched counterparts; S1P-enriched small, dense HDL3b and 3c provided slightly weaker effects (annexin V binding was reduced by 23 and 9% respectively), consistent with their lower levels of S1P-enrichment.

### CONCLUSIONS

The present lipidomic investigations have revealed that small HDL3 particles are enriched in S1P but poor in SM; the S1P/SM molar ratio increased 4.3-fold from HDL2b to HDL3c. Moreover, small HDL3 potently attenuated apoptosis in endothelial cells and delayed LDL oxidation. The S1P/SM molar ratio was strongly positively correlated with the antiapoptotic and antioxidative activities of HDL subfractions, thereby identifying elevated S1P/SM ratio as an integral feature of antiatherogenic small HDL3 particles. Published studies have documented subnormal levels of HDL cholesterol and apoA-I in patients with premature cardiovascular disease. In addition, recent data have demonstrated that the lipid composition of atheroprotective HDL particles is abnormal in such patients and particularly in subjects with type 2 diabetes and metabolic syndrome. These observations, in combination with the results obtained in the present study, indicate that apoA-I, S1P and SM levels may be abnormal in HDL subfractions in patients affected with a cardiovascular disease and/or dyslipidemia. Reduced levels of apoA-I, elevated levels of SM and/or reduced levels of S1P in small HDL3 should therefore reflect elevated cardiovascular risk. Vice versa, elevated levels of apoA-I, reduced levels of SM and/or elevated levels of S1P in small HDL3 should be associated with protection from cardiovascular disease. Consistent with this conclusion, our results indicate reduced levels of apoA-I in HDL3 from patients with Metabolic Syndrome and elevated levels of SM in HDL3 from patients with Type 2 diabetes. Combined determination of apoA-I, SM and S1P in HDL therefore provides information relevant both to the HDL cardioprotective function and cardiovascular risk.

TABLE 2
Molar contents of cholesteryl esters, free cholesterol and phospholipid subclasses in HDL particle
subfractions from normolipidemic subjects

| | Composition of HDL subspecies (mol/mol HDL) | | | | |
|---|---|---|---|---|---|
| Lipid component | HDL2b | HDL2a | HDL3a | HDL3b | HDL3c |
| CE | 104.50 ± 38.10^{n,o} | 105.80 ± 30.20^{n,o} (37.9) | 86.85 ± 18.74^{i,o} (41) | 42.38 ± 14.18^{h,j,,k,l} | 29.27 ± 7.18^{i,k,l,m} |
| | (39.9) | | | (42.6) | (50.5) |
| FC | 21.38 ± 5.34m^{m,n,o} | 14.76 ± 3.66^{m,n,o} (5.3) | 9.23 ± 1.34^{k,l,n,o} (4.4) | 4.13 ± 0.86^{k,l,m,o} (4.2) | 2.46 ± 0.34^{k,l,m,n} (4.2) |
| | (8.2) | | | | |
| Phosphatidylcholine | 88.56 ± 33.40^{n,o} | 114.00 ± 41.60^{n,o} (40.8) | 81.60 ± 16.58^{n,o} (38.5) | 36.62 ± 6.92^{o,k,l,m} | 20.25 ± 2.49^{k,l,m,n} |
| | (33.8) | | | (36.8) | (34.9) |
| Phosphatidylethanolamine | 2.47 ± 0.98^{n,o} (0.9) | 2.35 ± 0.49^{n,o} (0.8) | 1.72 ± 0.22^{o} (0.8) | 1.09 ± 0.68^{a,b} (1.0) | 0.44 ± 0.15^{k,l,m} (0.7) |
| SM | 33.63 ± 6.02^{n,o} (12.8) | 30.25 ± 7.72^{n,o} (10.8) | 21.91 ± 2.96^{n,o} (10.3) | 9.54 ± 1.76^{o,k,l,m} (9.6) | 3.57 ± 1.34^{k,l,m,n} (6.2) |
| Phosphatidylinositol | 9.76 ± 2.04^{n,o} (3.7) | 10.63 ± 1.16^{n,o} (3.8) | 9.35 ± 2.23^{o} (4.4) | 5.05 ± 2.03^{e,k,l} (5.1) | 1.65 ± 0.41^{d,k,l,m} (2.8) |
| Lysophosphatidylcholine | 1.44 ± 0.62^{n,o} (0.6) | 1.47 ± 0.58^{n,o} (0.5) | 1.24 ± 0.21^{d,o} (0.6) | 0.58 ± 0.17^{c,k,l} (0.6) | 0.33 ± 0.19^{k,l,m} (0.6) |

Data are shown for healthy male donors (n = 4). The % composition of the lipid moiety of each HDL particle subfraction as measured by HPLC (CE, FC, PC, phosphatidylethanolamine, SM, phosphatidylinositol and lysophosphatidylcholine but excluding TG and partial glycerides) is given in parentheses.
Superscripts are as follows:
^{a}p < 0.05 vs. HDL2b,
^{b}p < 0.05 vs. HDL2a,
^{c}p < 0.05 vs. HDL3a,
^{d}p < 0.05 vs. HDL3b,
^{e}p < 0.05 vs. HDL3c,
^{f}p < 0.01 vs. HDL2b,
^{g}p < 0.01 vs. HDL2a,
^{h}p < 0.01 vs. HDL3a,
ⁱp < 0.01 vs. HDL3b,
^{j}p < 0.01 vs. HDL3c,
^{k}p < 0.001 vs. HDL2b,
^{l}p < 0.001 vs. HDL2a,
^{m}p < 0.001 vs. HDL3a,
ⁿp < 0.001 vs. HDL3b,
^{o}p < 0.001 vs. HDL3c.

### REFERENCES

Ashen & Blumenthal, 2005 M.D. Ashen and R. S. Blumenthal, Clinical practice. Low HDL cholesterol levels, N Engl J Med 353 (2005), pp. 1252-1260.
Asztalos B F, Sloop C H, Wong L, Roheim P S. Two-dimensional electrophoresis of plasma lipoproteins: recognition of new apo A-I-containing subpopulations. Biochim Biophys Acta. 1993; 1169:291-300.
Bagdade J D, Buchanan W F, Pollare T, Lithell H. Abnormal lipoprotein phospholipid composition in patients with essential hypertension. Atherosclerosis. 1995; 117:209-215.
Barter P J, Clay M A, Rye K A. High density lipoproteins: the anti-atherogenic fraction. In: Barter P J, Rye K A, eds. Plasma Lipids and Their Role in Disease. Amsterdam: Harwood Academic Publishers; 1999.
Bligh E G, Dyer W J. A rapid method of total lipid extraction and purification. Can J Biochem Physiol. 1959; 37:911-917.
Bustos P, Ulloa N, Calvo C, Muller D, Duran D, Martinez J, Salazar L, Quiroga A. Monoclonal antibodies to human apolipoproteins: application to the study of high density lipoprotein subpopulations. Clin Chim Acta. 2000; 299:151-167.
Chancharme L, Therond P, Nigon F, Lepage S, Couturier M, Chapman M J. Cholesteryl ester hydroperoxide lability is a key feature of the oxidative susceptibility of small, dense LDL. Arterioscler. Thromb. Vasc. Biol. 1999; 19:810-820.
Chancharme L, Therond P, Nigon F, Zarev S, Mallet A, Bruckert E, Chapman M J. LDL particle subclasses in hypercholesterolemia. Molecular determinants of reduced lipid hydroperoxide stability. J Lipid Res. 2002; 43:453-462.

Chapman M J, Goldstein S, Lagrange D, Laplaud P M. A density gradient ultracentrifugal procedure for the isolation of the major lipoprotein classes from human serum. J. Lipid Res. 1981; 22:339-358.
Chapman M J. Therapeutic elevation of HDL-cholesterol to prevent atherosclerosis and coronary heart disease. Pharmacol Ther. 2006; 111:893-908.
Cheung M C, Albers J J. Distribution of cholesterol and apolipoprotein A-I and A-II in human high density lipoprotein subfractions separated by CsCl equilibrium gradient centrifugation: evidence for HDL subpopulations with differing A-I/A-II molar ratios. J Lipid Res. 1979; 20:200-207.
Cheung M C, Segrest J P, Albers J J, Cone J T, Brouillette C G, Chung B H, Kashyap M, Glasscock M A, Anantharamaiah G M. Characterization of high density lipoprotein subspecies: structural studies by single vertical spin ultracentrifugation and immunoaffinity chromatography. J. Lipid Res. 1987; 28:913-929.
Curtiss L K, Bonnet D J, Rye K A. The conformation of apolipoprotein A-I in high-density lipoproteins is influenced by core lipid composition and particle size: a surface plasmon resonance study. Biochemistry. 2000; 39:5712-5721.
Dupuy A M, Lehmann S, Cristol J P. Protein biochip systems for the clinical laboratory. Clin Chem Lab Med. 2005; 43:1291-1302.
Duriez P, Fruchart J C. High-density lipoprotein subclasses and apolipoprotein A-I. Clin Chim Acta. 1999; 286:97-114.
Escargueil-Blanc I, Meilhac O, Pieraggi M T, Arnal J F, Salvayre R, Negre-Salvayre A. Oxidized LDLs induce massive apoptosis of cultured human endothelial cells through a calcium-dependent pathway. Prevention by aurintricarboxylic acid. Arterioscler Thromb Vasc Biol. 1997; 17:331-339.
Esterbauer H, Striegl G, Puhl H, Rotheneder M. Continuous monitoring of in vitro oxidation of human low density lipoprotein. Free Radic. Res. Commun. 1989; 6:67-75.
Fielding P E, Kawano M, Catapano A L, Zoppo A, Marcovina S, Fielding C J. Unique epitope of apolipoprotein A-I expressed in pre-beta-1 high-density lipoprotein and its role in the catalyzed efflux of cellular cholesterol. Biochemistry. 1994; 33:6981-6985.
Folch J, Lees M, Sloane Stanley G H. A simple method for the isolation and purification of total lipides from animal tissues. J Biol Chem. 1957; 226:497-509.
Gowri M S, Van der Westhuyzen D R, Bridges S R, Anderson J W. Decreased protection by HDL from poorly controlled type 2 diabetic subjects against LDL oxidation may be due to the abnormal composition of HDL. Arterioscler Thromb Vasc Biol. 1999; 19:2226-2233.
Guerin M, Egger P, Le Goff W, Soudant C, Dupuis R, Chapman M J. Atorvastatin reduces postprandial accumulation and cholesteryl ester transfer protein-mediated remodeling of triglyceride-rich lipoprotein subspecies in type IIb hyperlipidemia. J Clin Endocrinol Metab. 2002; 87:4991-5000.
Guerin M, Le Goff W, Lassel T S, Van Tol A, Steiner G, Chapman M J. Atherogenic role of elevated C E transfer from HDL to VLDL(1) and dense LDL in type 2 diabetes: impact of the degree of triglyceridemia. Arterioscler Thromb Vasc Biol. 2001; 21:282-288.
Hansel B, Giral P, Nobecourt E, Chantepie S, Bruckert E, Chapman M J, Kontush A. Metabolic syndrome is associated with elevated oxidative stress and dysfunctional dense high-density lipoprotein particles displaying impaired antioxidative activity. J Clin Endocrinol Metab, 2004; 89:4963-4971.
Hanshaw R G, Smith B D. New reagents for phosphatidylserine recognition and detection of apoptosis. Bioorg Med. Chem. 2005; 13:5035-5042.
Holopainen J M, Medina O P, Metso A J, Kinnunen P K. Sphingomyelinase activity associated with human plasma low density lipoprotein. J Biol. Chem. 2000; 275:16484-16489.
Jain K K. Applications of biochip and microarray systems in pharmacogenomics. Pharmacogenomics. 2000; 1:289-307.
Kontush A, Chantepie S, Chapman M J. Small, dense HDL particles exert potent protection of atherogenic LDL against oxidative stress. Arterioscler Thromb Vasc Biol. 2003; 23:1881-1888.
Kontush A, Chapman M J. Functionally defective HDL: A new therapeutic target at the crossroads of dyslipidemia, inflammation and atherosclerosis. Pharmacol. Rev. 2006; 3:342-374.
Kontush A, de Faria E C, Chantepie S, Chapman M J. A normotriglyceridemic, low HDL-cholesterol phenotype is characterised by elevated oxidative stress and HDL particles with attenuated antioxidative activity. Atherosclerosis. 2005; 182:277-285.
Li Z, McNamara J R, Ordovas J M, Schaefer E J. Analysis of high density lipoproteins by a modified gradient gel electrophoresis method. J Lipid Res. 1994; 35:1698-1711.
Marcel Y L, Jewer D, Leblond L, Weech P K, Milne R W. Lipid peroxidation changes the expression of specific epitopes of apolipoprotein A-I. J Biol Chem. 1989; 264:19942-19950.
McVicar J P, Kunitake S T, Hamilton R L, Kane J P. Characteristics of human lipoproteins isolated by selected-affinity immunosorption of apolipoprotein A-I. Proc Natl Acad Sci USA. 1984; 81:1356-1360.
Mendez A J, Oram J F, Bierman E L. Protein kinase C as a mediator of high density lipoprotein receptor-dependent efflux of intracellular cholesterol. J Biol. Chem. 1991 Jun. 5; 266(16):10104-11.
Miyazaki O, Kobayashi J, Fukamachi I, Miida T, Bujo H, Saito Y. A new sandwich enzyme immunoassay for measurement of plasma pre-beta1-HDL levels. J Lipid Res. 2000; 41:2083-2088.
Nakamura Y, Kotite L, Gan Y, Spencer T A, Fielding C J, Fielding P E. Molecular mechanism of reverse cholesterol transport: reaction of pre-beta-migrating high-density lipoprotein with plasma lecithin/cholesterol acyltransferase. Biochemistry. 2004; 43:14811-14820.
Nobecourt E, Jacqueminet S, Hansel B, Chantepie S, Grimaldi A, Chapman M J, Kontush A. Defective antioxidative activity of small, dense HDL particles in type 2 diabetes: Relationship to elevated oxidative stress and hyperglycemia. Diabetologia. 2005; 48:529-538.
Nofer J R, van der Giet M, Tolle M, Wolinska I, von Wnuck Lipinski K, Baba H A, Tietge U J, Godecke A, Ishii I, Kleuser B, Schafers M, Fobker M, Zidek W, Assmann G, Chun J, Levkau B. HDL induces NO-dependent vasorelaxation via the lysophospholipid receptor S1P3. J Clin Invest. 2004; 113:569-581.
Pastier D, Dugue S, Boisfer E, Atger V, Tran N Q, van Tol A, Chapman M J, Chambaz J, Laplaud P M, Kalopissis A D. Apolipoprotein A-II/A-I ratio is a key determinant in vivo of HDL concentration and formation of pre-beta HDL containing apolipoprotein A-II. Biochemistry. 2001; 40:12243-12253.
Rader D J. Molecular regulation of HDL metabolism and function: implications for novel therapies. J Clin Invest. 2006; 116:3090-3100
Rainwater DL. Electrophoretic separation of LDL and HDL subclasses. Methods Mol. Biol. 1998; 110:137-151.
Rumsey S C, Stucchi A F, Nicolosi R J, Ginsberg H N, Ramakrishnan R, Deckelbaum R J. Human plasma LDL cryopreserved with sucrose maintains in vivo kinetics indistinguishable from freshly isolated human LDL in cynomolgus monkeys. J Lipid Res. 1994; 35:1592-1598.
Stoffel W. Sphingolipids. Annu Rev Biochem. 1971; 40:57-82.
Strejan G H, Smith P M, Grant C W, Surlan D. Naturally occurring antibodies to liposomes. I. Rabbit antibodies to sphingomyelin-containing liposomes before and after immunization with unrelated antigens. J. Immunol. 1979; 123:370-378.
Therond P, Abella A, Laurent D, Couturier M, Chalas J, Legrand A, Lindenbaum A. In vitro study of the cytotoxicity of isolated oxidized lipid low-density lipoproteins fractions in human endothelial cells: relationship with the glutathione status and cell morphology. Free Radic Biol Med. 2000; 28:585-596.
Tsimihodimos V, Karabina S A, Tambaki A P, Bairaktari E, Goudevenos J A, Chapman M J, Elisaf M, Tselepis A D. Atorvastatin preferentially reduces LDL-associated platelet-activating factor acetylhydrolase activity in dyslipidemias of type IIA and type IIB. Arterioscler Thromb Vasc Biol. 2002; 22:306-311.
Valabhji J, McColl A J, Schachter M, Dhanjil S, Richmond W, Elkeles R S. High-density lipoprotein composition and paraoxonase activity in Type I diabetes. Olin Sci (Lond). 2001; 101:659-670.
Visentin B, Vekich J A, Sibbald B J, Cavalli A L, Moreno K M, Matteo R G, Garland W A, Lu Y, Yu S, Hall H S, Kundra V, Mills G B, Sabbadini R A. Validation of an anti-sphingosine-1-phosphate antibody as a potential therapeutic in reducing growth, invasion, and angiogenesis in multiple tumor lineages. Cancer Cell. 2006; 9:225-238.
Wang T J, Gona P, Larson M G, Tofler G H, Levy D, Newton-Cheh C, Jacques P F, Rifai N, Selhub J, Robins S J, Benjamin E J, D'Agostino R B, Vasan R S (2006). “Multiple biomarkers for the prediction of first major cardiovascular events and death”. N. Engl. J. Med. 355 (25): 2631-9.
Weinberger S R, Morris T S, Pawlak M. Recent trends in protein biochip technology. Pharmacogenomics. 2000; 1:395-416.
World Health Organization (2004) The atlas of heart disease and stroke, World Health Organization, Geneva, Switzerland.
Zhang H, Desai N N, Olivera A, Seki T, Brooker G, Spiegel S. Sphingosine-1-phosphate, a novel lipid, involved in cellular proliferation. J Cell Biol. 1991; 114:155-167.

1. A method for assessing the risk of a cardiovascular disease and/or monitoring the therapeutic outcome of a patient following a therapy for treating a cardiovascular disease in a patient, said method comprising:
a) isolating in a blood sample obtained from said patient the high density lipoprotein (HDL) fraction or a subfraction thereof; and
b) measuring in said HDL fraction or subfraction thereof the concentration of one or more biomarkers selected from the group consisting of Sphingosine-1-Phosphate (S1P), sphingomyelin (SM) and Apolipoprotein A-I (apoA-I).

2. A method according to claim 1, wherein the cardiovascular disease is atherosclerosis.

3. A method for diagnosing a dyslipidemia in a patient and/or monitoring the therapeutic outcome of a patient following a therapy for treating a dyslipidemia, said method comprising:
a) isolating in a blood sample obtained from said patient the high density lipoprotein (HDL) fraction or a subfraction thereof; and
b) measuring in said HDL fraction or subfraction thereof the concentration of one or more biomarkers selected from the group consisting of Sphingosine-1-Phosphate (S1P), sphingomyelin (SM) and Apolipoprotein A-I (apoA-I).

4. A method according to any of claim 3, wherein said biomarker is S1P.

5. A method according to any of claim 3 wherein said one or more biomarkers are S1P and SM.

6. The method according to any of claim 3, wherein said HDL subfraction is the HDL3 subfraction.

7. The method according to claim 6 wherein said HDL3 subfraction is the HDL3c subfraction.

8. The method according to claim 3, wherein said biological sample is a plasma or a serum sample.

9. The method according to claim 3, wherein said method is performed with an array chip.

10. (canceled)

11. A kit comprising at least 2 binding partners selected from the group consisting of a binding partner which selectively interacts with apoA-I, a binding partner which selectively interacts with S1P and a binding partner which selectively interacts with SM.

12. A kit comprising at least 2 antibodies selected from the group consisting of an anti-apoA-I antibody, an anti-S1P antibody and an anti-SM antibody.

13. The method according to claim 1, wherein said biomarker is S1P.

14. The method according to claim 1, wherein said one or more biomarkers are S1P and SM.

15. The method according to claim 1, wherein said HDL subfraction is the HDL3 subfraction.

16. The method according to claim 5, wherein said HDL3 subfraction is the HDL3c subfraction.

17. The method according to claim 1, wherein said biological sample is a plasma or a serum sample.

18. The method according to claim 1, wherein said method is performed with an array chip.
